SEQUENCE LISTING (1 sequence)

SEQ ID NO: 1 - DNA, 37 bp, Artificial Sequence
  Artificial sequence generated as an example
cgtagcggcgtaaattctatacgtagcggcgtaaatt

(19) United States
(12) Patent Application Publication
(10) Pub. No.: US 2018/0314795 A1
(43) Pub. Date: Nov. 1, 2018

(54) INTERACTIVE PRECISION MEDICINE EXPLORER FOR GENOMIC ABBERATIONS AND TREATMENT OPTIONS

(71) Applicant: KONINKLIJKE PHILIPS N.V., EINDHOVEN (NL)

(72) Inventors: Yee Him Cheung, Boston, MA (US); Nevenka Dimitrova, Pelham Manor, NY (US); Johanna Maria de Bont, Eindhoven (NL)

(21) Appl. No.: 15/964,180

(22) Filed: Apr. 27, 2018

Related U.S. Application Data
(60) Provisional application No. 62/490,921, filed on Apr. 27, 2017.

(51) Int. Cl. G06F 19/26; G06F 19/22; G06F 19/18; G06F 17/24; G06F 3/0481; G16H 10/60; G16H 50/50

(52) U.S. Cl. CPC G06F 19/26; G06F 19/22; G06F 19/18; G16H 50/50; G06F 3/0481; G16H 10/60; G06F 17/241

(57) ABSTRACT

A data-driven integrative visualization system and method for summarizing and presenting genomic aberrations, their drug responses and multi-omic data of a patient, is disclosed. Specifically, a method for displaying genomic aberrations and multi-omic data of a patient in an interactive tool which allows the medical practitioner to access underlying supporting biologic and scientific evidence from relevant knowledge bases through a set of graphical interactions, is described. The method comprises the steps of obtaining and inputting multi-omic data of a patient or cohorts, identifying genomic aberrations and their drug responses, and displaying this information in a first level interactive classical/circular ideogram in one or multiple layers on a GUI, from which the user can access and view further information on the gene and molecular levels. The system provides an improved process of integrative analysis on a patient's multi-omic data for effective treatment planning.

### CROSS-REFERENCE TO RELATED APPLICATIONS

This application claims priority to U.S. provisional patent application No. 62/490,921, filed on Apr. 27, 2017, the entire disclosure of which is hereby incorporated by reference herein for all purposes.

### FIELD OF THE INVENTION

The present invention relates to a data-driven integrative visualization system and method for summarizing and presenting genomic aberrations, their drug responses and multi-omic data of a patient. Specifically, a method for displaying genomic aberrations and multi-omic data of a patient in an interactive tool which allows the medical practitioner to access underlying supporting biologic and scientific evidence from relevant knowledge bases through a set of graphical interactions, is described. The method comprises the steps of obtaining and inputting multi-omic data of a patient or cohorts, identifying genomic aberrations and their drug responses, and displaying this information in a first level interactive classical/circular ideogram located by genome coordinates in one or multiple layers on a GUI, from which the user can access and view further information on the gene and molecular levels. The system provides an improved process of integrative analysis of a patient's multi-omic data for effective treatment planning.

### BACKGROUND OF THE INVENTION

Idiogram is a standard visual tool for locating the positions of individual genes or aberrations on chromosomes. Traditionally, the prominent Giemsa-staining bands are marked on each chromosome and they are named following the International System for Cytogenetic Nomenclature (ISCN). In the ISCN scheme, chromosomes are assigned a short arm and a long arm, which begin with the designations p and q respectively. The numbering for a chromosome begins at its centromere and the numbers assigned to each region increase towards the telomere.

Krzywinski, M. et al., Circos: an information aesthetic for comparative genomics, Genome Research 19, 1639-1645 (2009), describe a software-driven tool for visualizing data and information in a circular format, which makes it ideal for exploring relationships and information. This format was originally designed for visualizing genomic data and for creating publication-quality infographics and illustrations, but is also applied in data fields to describe the relationships between objects or positions in a circular layout, and to summarize multilayered annotations of one or more scales. When used in genomics as an alternative to classical ideograms, the circular genome coordinates make it effective in displaying variations in genomic structure, and data as scatter, line and histogram plots, heat maps, tiles, connectors and texts in multiple tracks. Currently, its use in genomics is mainly for the static presentation of cohort data, most often in scientific publications. It neither supports user interaction or data exploration, nor facilitates sample/cohort comparison, and is not intended for presenting precision medicine or clinical trial information for an individual patient.

The goal of this invention is to create a new tool that is useful for precision medicine software applications, such that both genomic aberrations and their corresponding treatment options and drug responses are summarized for one or more patients. The existing notion of the classical idiogram or circos plot is fairly simple, and non-interactive. However, by creating a new representation that is interactive, we enable users to navigate and view the details of the genomic data at different levels, explore the underlying scientific evidence and have quick access to relevant information in knowledge-bases. The new interactive Precision Medicine Explorer of this invention significantly improves the process of integrative analysis of a patient's multi-omic data for effective treatment planning.

In further contrast to prior art, this invention is an effective precision medicine tool for summarizing and presenting the genomic aberrations, their drug responses and multi-omic data of a patient. It facilitates the understanding of the underlying biology and the supporting scientific evidence by allowing a user to dig deep into the details and access relevant information from knowledge bases, such as ClinVar (www.ncbi.nlm.nih.gov/clinvar/), LOVD

(Leiden Open (source) Variation Database—www.lovd.nl/3.0/home), HGMD Human Gene Mutation Database www.hgmd.cf.ac.uk/ac/index.php, COSMIC cancer.sanger.ac.uk/cosmic, 1000 Genomes www.internationalgenome.org, OMIM omim.org and other databases, through an extensive set of graphical interactions.

Our Precision Medicine Explorer can be implemented as a standalone application or a GUI component that takes processed omic data as inputs. The software can run as software, as a service on a cloud based infrastructure, or as a standalone application on a mobile device, laptop or local server. Each layer is associated with an independent data environment, which may include multiple tables for mutations (SNVs, indels, CNVs, fusions, etc.) with annotation information, drug options, clinical trials, gene/exon expressions, and methylation. Besides visualizing and presenting the data, the tool also handles user inputs and interactions, and queries different knowledge bases to incorporate further information if necessary.

### SUMMARY OF THE INVENTION

It is an object of the present invention to provide an improved presentation for exploration of patient-oriented omic data (genomic, transcriptomic, proteomic, epigenomic, etc.), treatment options and underlying scientific evidence for use by clinicians, oncologists, geneticists, medical professionals and scientists. In particular, it is an object of the present invention to provide a system and method that solves the above-mentioned problems of the prior art by providing an interactive visualization tool for summarizing and presenting patient multi-omic data in a circular or linear multilayered format. It is also an object of the present invention to provide a system and method for providing patient genomic aberration, detailed annotations and related drug response data to improve the view of combined effects of multiple genomic aberrations on the functional effect as well as link to potential therapy. It is a further object of the present invention to provide interactive access, through the visual multi-omics format, to underlying intergenic genomic information, methylation and gene/exon expression data, on a genic scale, and nucleotide sequence, amino acid sequence and methylation data, on a molecular scale. It is also an object of the present invention to provide an alternative to the prior art.

Thus, the above-described object and several other objects are intended to be obtained in a first aspect of the invention by providing a system and method for providing relevant patient-specific genomic information, such system and method comprising:

obtaining genomic aberration and other omics data from a patient and storing said data on a non-transitory computer readable storage medium—one of the common processses for data generation involves the collection of tissue and blood samples from the patient, performing next-generation sample preparation and DNA/RNA seqeuncing, read alignment and calling of variants and gene expressions;

optionally selecting a cohort of samples based on user-defined demographic and phenotypic criteria from a repository of patient or healthy samples, and extracting their genomic aberration and omics data for comparison with the patient of interest;

annotating the genomic aberration and omics data using internal/external knowledge bases, which include information such as mutation impact, population allele frequency, disease association with model of inheritance, drug response, etc.

filtering the genomic aberrations and omics data based on user-defined criteria, such as chromosome regions, genes, variant type/function/impact/population allele frequency, etc.

with a computing device with a graphical user interface, displaying the genomic aberration and omics data in an interactive multi-level format, which comprises;

a first level (Level 1), comprising an interactive chromosomal view that summarizes all the clinically relevant or actionable genomic aberrations of a patient by marking them on the genome coordinates, including known drug responses associated with a particular mutation/gene marked next to the mutation/gene accordingly, the first level further comprising two additional levels which can be accessed by the user which include Level 1A, a circular ideogram view where chromosomes are arranged in a circular layout, and Level 1B, an ideogram view, where each chromosome is separately displayed in a schematic;

a second level (Level 2), comprising an interactive intergenic genomic scale where multiple genes are displayed with their expression levels indicated by color. Additional Data tracks can be included to add more details such as methylation, chromatin immunoprecipitation sequencing (ChIP-Seq), Native Elongating Transcripts Sequencing (NET-Seq) and Assay of Transposase Accessible Chromatin Sequencing (ATAC-Seq) data at any view levels which may improve the functional view of genomic aberrations; With ChIP data we will see if there is functional binding of the transcription factors to their targets; with NET-Seq we can analyze the genome-wide transcriptional activity; and with ATAC-Seq we can study chromatin accessibility. These aspects may lead to conclusions about activation of gene targets downstream.

a third level (Level 3), comprising an interactive genic scale, depicting the structure and functional blocks within a gene, omics data such as methylation levels and gene/exon expression, the 3D protein structure (ribbon plot) with mutations marked and including general information about the gene; and

a fourth level (Level 4), comprising a molecular scale displaying the molecular sequence and its detailed annotations, such as the nucleotide sequence of the reference genome, the corresponding amino acid sequence in the protein-coding regions, nucleotide/amino acid changes caused by the mutations, exon/gene expression, methylation levels of CpG sites, ChIP-Seq data for histone modification, and any additional data tracks that incorporate more details. The complete human reference sequence (GRCh37) can be downloaded in fasta format from the UCSC Genome Browser Server (hgdownload.cse.ucsc.edu/goldenPath/hg19/bigZips/) and the exon locations of the known canonical genes and other gene annotations can also be downloaded from the UCSC Genome Browser; and

displaying said first through forth levels individually on a graphical user interface.

By clicking/selecting a region on the chromosome or specifying a range of chromosome positions, users can view, access and explore data at these different view levels. The data come from different sources: (i) the patient-specific data such as mutations, gene expressions and additional data tracks can be stored as flat files or database tables, (ii) the variant annotations can be retrieved from local or online knowledge-bases, (iii) the reference genomes and gene locations and annotations consist of data files that can be downloaded from public repositories and stored locally.

In addition, a second aspect of the present invention is directed to a display of the omics data of a patient or a cohort of patients in multiple layers for side-by-side comparison. The genome coordinates are locked and in line across layers. Users are able to add/remove/combine/change the order of multiple layers and explore any one of them in details through all interactions that are applicable to a single layer, which when executed by a computing device with a graphical user interface, cause the device to carry out the steps of the method as described above.

### BRIEF DESCRIPTION OF THE DRAWINGS

The methods according to the invention will now be described in more detail with regard to the accompanying figures. The figures showing ways of implementing the present invention and are not to be construed as being limiting to other possible embodiments falling within the scope of the attached claims.

The methods according to the invention will now be described in more detail with regard to the accompanying figures. The figures showing ways of implementing the present invention and are not to be construed as being limiting to other possible embodiments falling within the scope of the attached claims.

FIG. 1 is a high-level flow diagram that gives an overview of the computational steps and data sources involved in the processing and presentation of multi-omics data in our Precision Medicine Explorer;

FIG. 2 is a flow diagram that shows the detailed steps and components for the two main functionalities of the Precision Medicine Explorer: (a) filtering and searching of variant and omics data, and (b) data visualization and exploration;

FIG. 3 is a circular ideogram view of Level 1, displaying the genomic aberrations of a patient and their associated drug responses;

FIG. 4 is a classical ideogram view of Level 1, displaying the genomic aberrations of a patient and their associated drug responses;

FIG. 5 is a view of Level 2, an intergenic genomic scale where multiple genes are displayed with their expression levels indicated by color;

FIG. 6 is a view of Level 3, a genic scale where the methylation and gene/exon expression levels are indicated by color;

FIG. 7 is a view of Level 4, showing the nucleotide sequence, amino acid sequence and methylation level;

FIG. 8 is a schematic view of multiple layers for the comparison of genomic aberrations and treatment options across different patients and cohorts;

FIG. 9 illustrates a circular ideogram showing genes with associated keywords for searching purposes; and

FIG. 10 is a 3D view of our Precision Medicine Explorer.

### DETAILED DESCRIPTION OF THE INVENTION

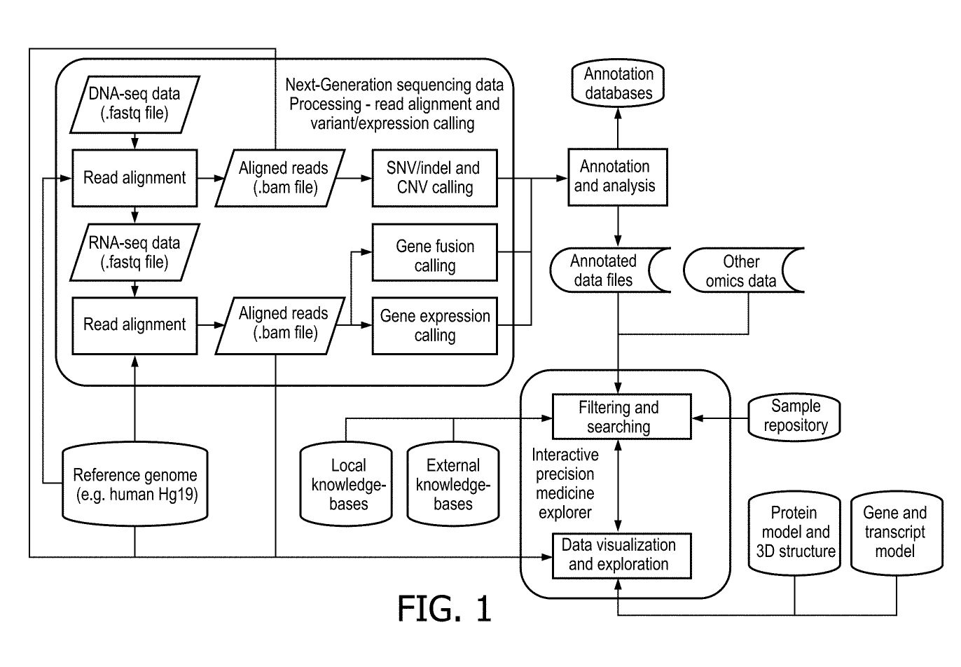

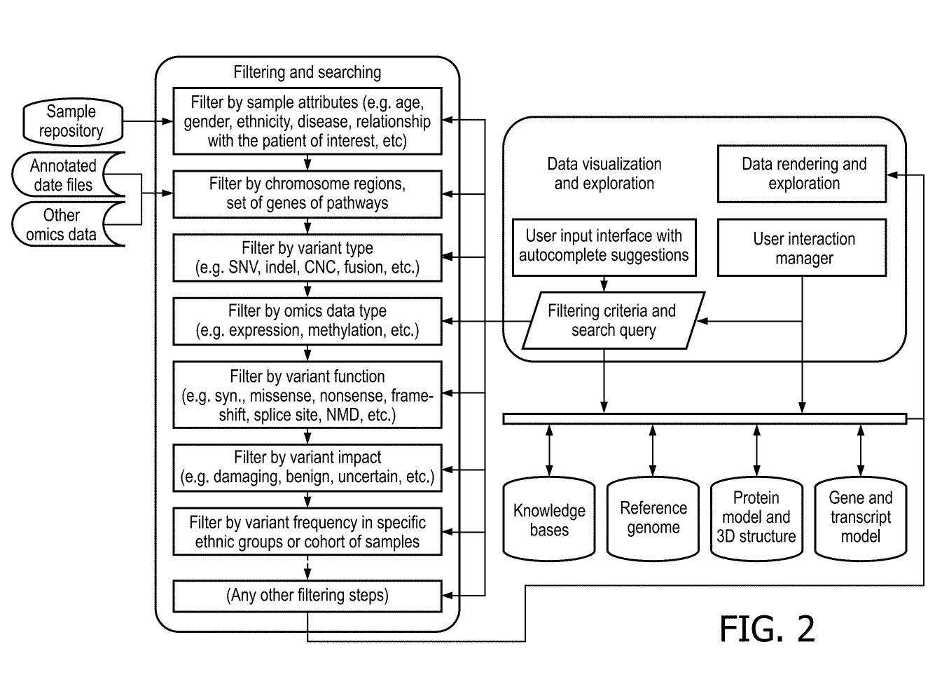

The present invention provides a system and method for summarizing and presenting genomic aberrations, their drug responses and multi-omic data of a patient, by displaying genomic aberrations and multi-omic data of the patient in an interactive classical/circular ideogram format which allows the medical practitioner to access underlying supporting biologic and scientific evidence from relevant knowledge bases through a set of graphical interactions. The present invention is described in further detail below with reference made to FIGS. 1-10. Referring now to the figures, FIG. 1 is a flow diagram that shows an overview of the computational steps and data sources involved in the processing and presentation of multi-omics data in the Precision Medicine Explorer. Similarly, FIG. 2 is a flow diagram showing the steps and components for two main functionalities of the Precision Medicine Explorer: (a) filtering and searching of variant and omics data, and (b) data visualization and exploration. FIGS. 1 and 2 illustrate an embodiment of the invention which provides a system and a method for obtaining and organizing relevant patient-specific genomic information, presenting such information on a visual display that is a circular or linear multilayered interactive plot, usually displayed on a graphical user interface. The method entails obtaining genomic aberration and other omics data from a patient and storing that data on a non-transitory computer readable storage medium. One of the common processses for data generation involves the collection of tissue and blood samples from the patient, performing next-generation sample preparation and DNA/RNA seqeuncing, read alignment and culling of variants and gene expressions, etc. Optionally, a user could select a cohort of samples based on demographic and phenotypic criteria, defined by the user, from a repository of patient or healthy samples, and extracting their genomic aberration and omics data for comparison with the patient of interest. The genomic aberration and omics data are annotated the using internal/external knowledge bases (FIG. 1), which include information such as mutation impact, population allele frequency, disease association with model of inheritance, drug response, etc. The genomic aberrations and omics data are then filtered based the on user-defined criteria (FIG. 2), such as chromosome regions, genes, variant type/function/impact/population allele frequency, etc.

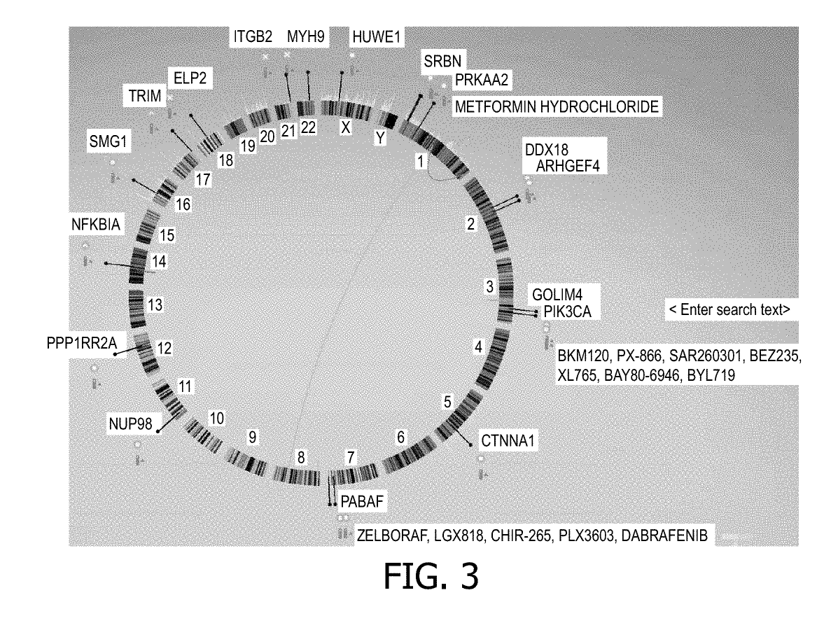

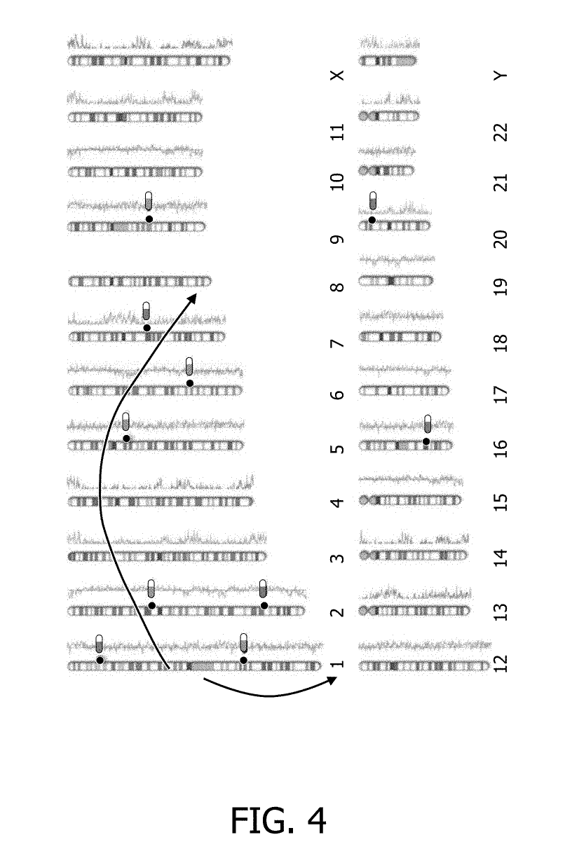

With a computing device having a graphical user interface, the genomic aberration and omics data are then displayed in an interactive multi-level format. At Level 1 of the method and system for displaying patient-specific genomic data and genomic aberrations, all the clinically relevant or actionable aberrations of a patient are summarized by marking them on the genome coordinates (see FIGS. 3 and 4). If there are any drug responses associated with a mutation/gene, they are marked next to the mutation/gene accordingly. Within this level, there are at least three possibilities: Level 1A—circular ideogram view, where chromosomes are arranged in a circular layout, and Level 1B—classical ideogram view, where each chromosome is separately displayed in a schematic that uses the familiar karyogram representation. According to an embodiment of the present invention, FIG. 3 is an interactive circular ideogram view at Level 1A and FIG. 4 is an interactive classical ideogram view at Level 1B. Both views are displayed by the computer on a graphical user interface (“GUI”). Users are able to switch from one view to the other by interacting with the GUI. A third representation would be linear horizontal representation which contains the same layers on a horizontal axis stacked on top of each other. The user accesses the chromosonal sub-levels by clicking on or selecting a mutation or gene in the GUI at Level 1, and by similarly selecting a region on the chromosome, the user can “zoom in” to view and explore data at different levels.

FIG. 5 illustrates the second level, Level 2 of the embodiment of FIGS. 3 and 4. Level 2 is an interactive intergenic genomic scale where multiple genes are labeled by their gene symbols and displayed with their expression levels indicated by color, along with any relevant/targetable mutations and their corresponding drug options. The user may add data tracks, such as methylation, ChIP-Seq, NET-Seq and ATAC-Seq data to incorporate more details to complete the functional picture of the genomic aberrations (or the lack thereof).

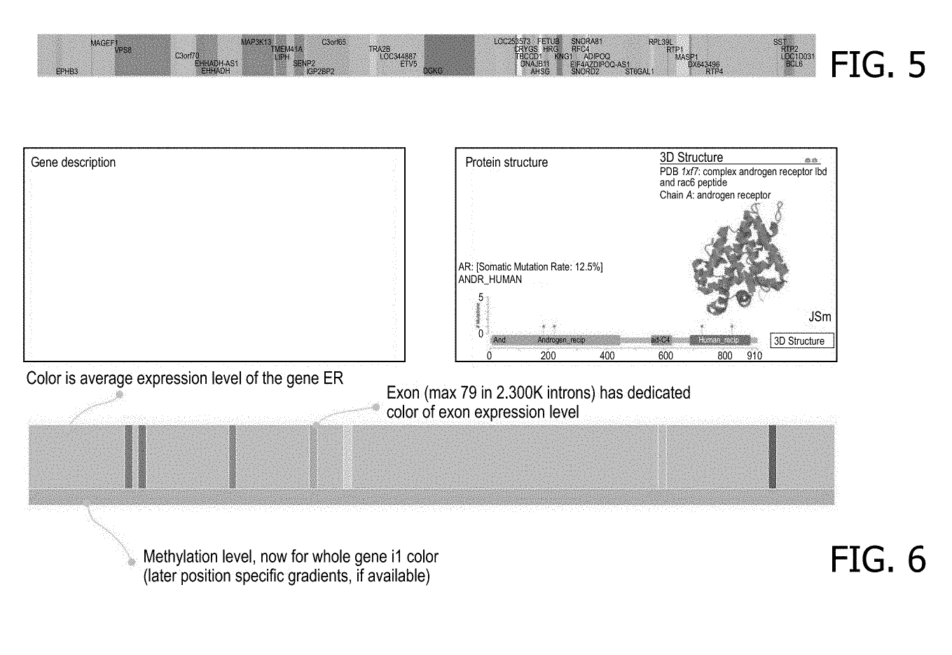

By selecting a specific gene at Level 2, the user is directed to Level 3 of this embodiment, as shown in FIG. 6. Level 3 is a genic scale where the methylation, gene/exon expression levels and other omics data of the gene selected at Level 2 are indicated by color or other attributes, along with any relevant/targetable mutations and their corresponding drug options. Further data tracks as already mentioned can be added to incorporate more details. The reason for this multi-track representation is to be able to make inferences about the functional impact of the genomic aberrations. With the multi-track representation, we want to support event-based querying where multiple events for the SAME gene may affect the ability of the gene to drive a tumor. We need to enable better association of drugs (e.g., ALK fusions to a targeted drug named Crizotinib—that may inhibit a gene with an activating mutation or avoid therapies that may be directed at inactivated genes). Level 3 also includes general information, included at the top for reference, about the gene selected, its functional blocks (promoter, transcription start/stop site, exon, intron, etc.) and 3D structure (ribbon plot) with mutations being marked.

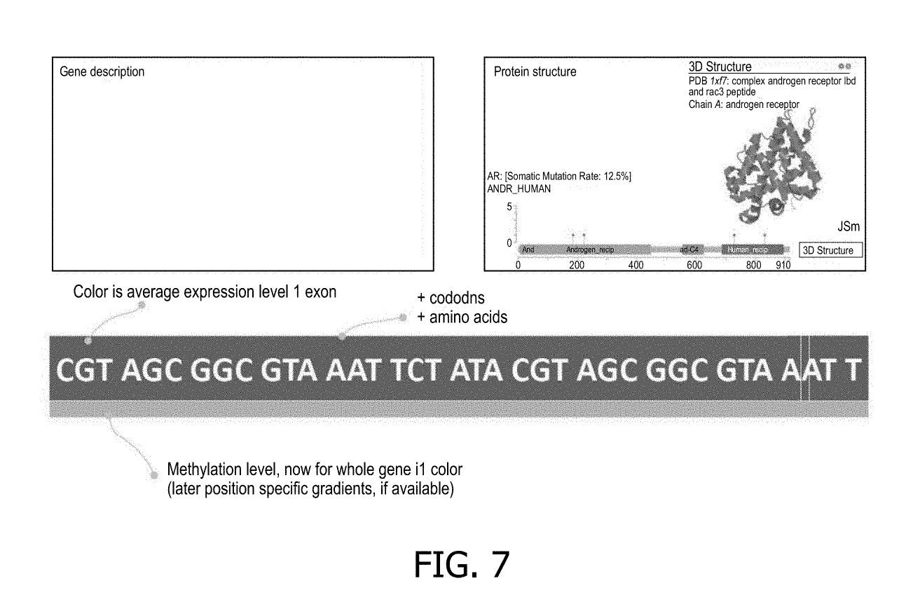

Similarly, the user accesses Level 4, as seen in FIG. 7, by selecting a specific gene at Level 3. Level 4 comprises information about the gene at the molecular level where the nucleotide sequence, amino acid sequence and methylation level are displayed. As aforementioned, data tracks can be added to incorporate more details, such as the nucleotide and amino acid changes caused by the mutations and create impression about the functional impact of the genomic aberrations. The important information that the user needs to visualize is if there is activating effect of the genomic aberrations: mutations/fusions on gene expression and downstream targets of that gene, or inactivating effect. By bringing this information together within the single visual framework, we bring the evidence so that the clinician is able to make decisions.

### Mutations and Drug Response

To enhance data presentation, the invention employs different symbols to represent different types of aberrations and drug/clinical trial associations with their levels of significance indicated by properties such as color and size, as can be seen in FIG. 3. An example of a scheme of data representation is as follows:
1. Single nucleotide variant (SNV)—A circle symbol for missense; A square symbol for nonsense
2. Insertion—A downward facing triangle symbol
3. Deletion—A cross or “X” symbol
4. Fusion—an arc joining the donor and acceptor genes
5. Copy number variation—a plus sign with the number of copies at the top right as a superscript (⊕³)
6. Over- or under-expression: An up arrow for over-expression and a down arrow for under-expression and the differential expression in log 2 fold change can be labelled at the top right
7. Variant classification, such as pathogenic, likely pathogenic, unknown significance (VUC), likely benign and benign, can be represented by different colors of the mutation symbol
8. A combined pathogenicity score based on multiple algorithms can be marked at the top right of the mutation symbol, e.g. a square symbol with a “0.9” superscript denotes a nonsense SNV with a combined pathogenicity score of 0.9
9. Additional annotations, such as frame shift (FS), splice site (SS), nonsense mediated decay (NMD), etc., can be labelled at the top right of the mutation symbol, e.g., a downward facing triangle with a “FS” superscript denotes a frameshift insertion
10. Each mutation is precisely labelled by using HGSV nomenclature (hgvs.org/mutnomen/). Additional nomenclatures may be used.
11. Explicit reference to activating or inactivating genomic aberration is made in the UX. This information can be inferred based on 1) the pathogenicity score, or 2) manually curated information that is assembled based on previous experimental and published findings.
12. Drugs option is represented by a pill
   (a) Drug options with increased response is denoted by a pill with an up-arrow in green
   (b) Drug options with decreased response is denoted by a pill with a down-arrow in blue
   (c) Drug options with severe side effects is denoted by a pill with an exclamation mark in red
   (d) The best level of evidence among the drug options is indicated by the fill level
   (e) Number of drug options belonging to the same category is labelled next to the symbol
   (f) For example, a green pill with a superscript including a chevron and “4” means there are four drugs with increased response associated with a mutation, whereas a red pill with a superscript including an exclamation point and “2” indicates there are two drugs with severe side effects, or if there is orange color, that means that the genomic aberration is associated with a resistant marker.
13. Clinical trial is represented by a test tube, with the number of trials stated at the top right and the level of evidence, if available, indicated by the fill level, e.g., a test tube with a “2” superscript indicates there are two clinical trials associated with a mutation
14. Symbols of the genes carrying clinically relevant mutations are marked at their genomic positions, with their mutations grouped and listed next to them.
15. The strand of a gene can be indicated by an arrow: →right or clockwise for forward strand, ←left or anti-clockwise for reverse strand
   The choice of symbols is not restricted to those illustrated in the above examples.

### Interactions

To enable the seamless navigation to the patient's multi-omic data at different levels of details and quick access to relevant information from different knowledge bases, the Precision Medicine tool of this invention is highly interactive and user friendly. The set of supported user interactions include, but are not limited to, the following:
1. Toggle between the classical ideogram, circos and horizontal (linear) views of the genome
2. Zoom in/out to different data levels by using a zoom slider, selecting a region on the genomic scale or directly specifying a gene, locus or the start and end chromosome positions
3. Rearrange the layout of chromosomes in the ideogram, rotate the circular ideogram or navigate to nearby regions by swiping
4. Select the inclusion/exclusion criteria for aberrations to be displayed, e.g., by specifying the types of mutations and the chromosome regions or gene subsets
5. Import and display additional tracks of data and annotation, e.g., mutational density
6. Select and display the omic data of one or more individual patients and cohorts in multiple layers
7. Hover on any color-scaled data, such as gene expression and methylation levels, and display the actual numerical values
8. Select a nucleotide, amino acid or mutation and their locations will be marked on the corresponding gene and 3D protein structure (see FIG. 7)
9. Rotate and zoom in/out the 3D protein structure
10. Select and display genes, mutations or other data associated with a concept or keywords
11. Access to more detailed information related to an object or component by clicking/hovering on it, or right-clicking and then selecting from a pop-up menu:
   (a) Mutations—chromosome/transcript/protein positions, amino acid changes, genotypes for germline mutations or variant allele fraction for somatic mutations, allelic balance, number of reads (for sequencing data), call quality (e.g., phred score), function (nonsense, missense, frame-shift, splice-site, NMD, etc.), variant classification, population allele frequencies, pathogenicity scores, related publications, etc.
   (b) Drugs options—a list of drug names, their levels of evidence, supporting publications, etc.
   (c) Clinical trials—a list of clinical trials, conducting institutions, short description, etc.
   (d) Gene-level details—full name of the gene, brief description, genomic size, number of exons, pathway/disease/drug associations, summary of patient-specific data such as gene expression and list of mutations, etc.
   (e) Information on the functional impact of the genomic aberrations
12. Include information on activating or deactivating effect of the genomic aberration
13. Include hyperlinks for terms, such as gene symbols and drug names, whenever necessary for further information

### Comparison of Multiple Samples and Cohorts

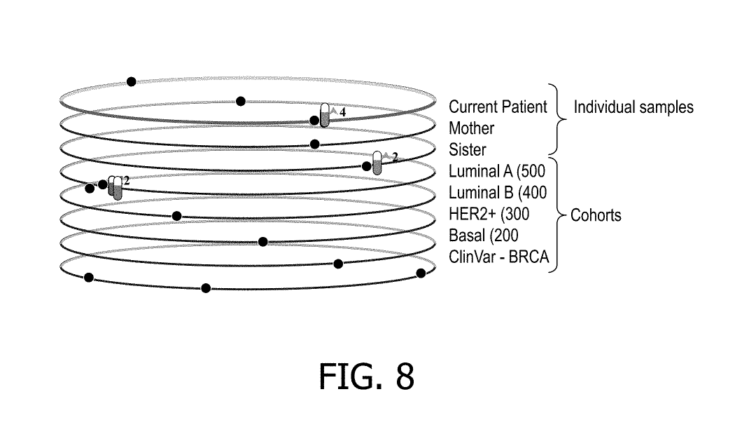

In a further embodiment, users can choose to display the omic data of a patient or a cohort of patients in multiple layers of the visual representation in the Precision Medicine Explorer for side-by-side comparison. See FIG. 8. The genome coordinates of each layer of ideogram should be coherently aligned with other layers. Users are able to add/remove/combine/change the order of multiple layers and explore any one of them in detail through all interactions applicable to a single layer. For example, FIG. 8 schematically illustrates a stack of circular layers for the comparison of genomic aberrations and treatment options across different patients and cohorts. Each layer presents the data of one patient or a cohort consisting of many patients. In this example, the genomic aberrations of the current patient are summarized in the top circle, and compared against individuals (the genomic profile of the patient's mother and sister), cohorts that have prognostic information (Luminal A, Luminal B, HER2+, Basal) and BRCA mutations from ClinVar.

### Presentation Filters for Genomics Aberrations

In genomics, it is customary to offer multiple filtering options to the user for each of the types of genomic aberrations. Within this embodiment, the goal is to associate the genomic aberrations to key evidence for treatment planning. In any embodiment of this invention, users can determine what data is to be presented in one or multiple layers of ideogram by applying a combination of filters that include but are not limited to the following:
1. Chromosome regions, e.g., chr1:1000000-5000000, chrX, etc.
2. Genes
   (a) List of specific genes
   (b) Biological concepts or terms that are associated with gene subsets, e.g., oncogene, suppressor, transcription factor, signaling pathways such as ER, PR, Wnt, PI3K, MAPK, etc.
   (c) Significantly mutated genes (SMGs)—users can select the methods for computing the SMGs and their parameters
   (d) Mutation burden—users can specify the number and types of mutations that a gene needs to carry to be included for display
   (e) Genes which have associated drug response information:
3. Variant Type: single nucleotide variants (SNVs), short insertions/deletions (indels), copy number variations (CNVs), gene fusions, over expression, under expression, etc.
4. Variant Function: synonymous, missense, nonsense, nonsense mediated decay (NMD), frameshift, splice site, promoter, etc.
5. Variant Impact
   (a) Therapeutic/Pharmacogenetic—variants with available drug options. Genomic aberrations have associated drug response information: 1) resistance association that depicts that the mutation is associated with resistance within a certain indication and 2) response association that depicts that the mutation is associated with likely response to the drug within a certain indication (e.g., response to First generation Tyrosine kinase inhibitor)
   (b) Classification—can be based on the ACMG guidelines, i.e., Classes 1-5 for somatic mutations, and for germline mutations “pathogenic,” “likely pathogenic,” “uncertain significance,” “likely benign” or “benign”
   (c) Pathogenicity prediction—users can choose a combination of algorithms and their thresholds, which are joined together by “and/or” operators
6. Variant Frequency in Ethnic Groups—minor allele frequency thresholds in one or more ethnic groups (white/black/Asian/all), with the conditions joined by “and/or” operators
7. Variant Frequency in Samples/Cohorts—for each sample/cohort, users can specify the range of the number/frequency of a variant or their carriers, with the conditions joined by “and/or” operators
   Depending on the purpose of the application, e.g. diagnostic, therapy selection or research, different default filter settings can be applied so that only the relevant information is shown.
   Search by Keywords with Autocomplete Suggestions

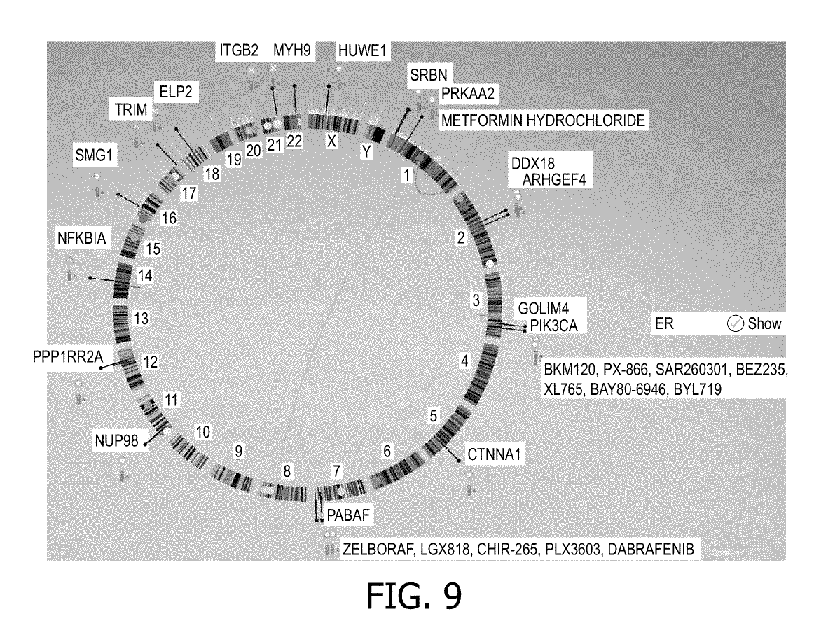

Users can show the genes or other information associated with a keyword on the ideogram by typing the keyword in a search box with autocomplete functionality. The search term can be a gene symbol, signaling pathway, disease, drug, or biological concept such as oncogene/suppressor, etc. Users can also search for a combination of these terms concatenated by logical operators, such as “,/OR”, “&/AND”, etc. Once the data related to the search term(s) are retrieved from the databases, they are displayed on the same or a separate ideogram (see FIG. 9). The search results can be highlighted and presented in such a way that they are distinguishable from the patient's primary data. Search history is tracked to let users select the results of one or more searches for quick viewing and comparison.

Referring to FIG. 9, a keyword search allows genes associated with a term to be looked up and displayed in the ideogram. In this example, all genes in the “ER Pathway” are shown.

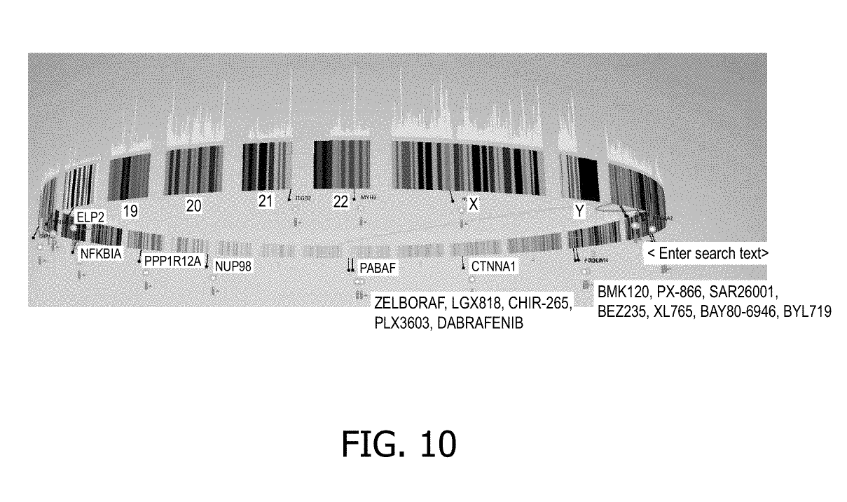

To make the zoom-in or zoom-out transition look continuous and smooth, and enhance the navigation and user experience, our Precision Medicine Explorer includes a 3D option that enables users to view the chromosome layouts from different visual perspectives (see FIG. 10).

Association with Evidence for Key Findings

One essential functionality of our Precision Medicine Explorer is to display the drugs/treatments with their known predicted/experimental/clinical responses (increased/decreased) or clinical trial options associated with patient-specific data, such as genomic aberrations, up/down-regulated gene expressions, abnormal methylation levels or other omics anomalies with supporting evidence, which can be further explored through user interactions. For example, the gene mutation BRAF V600E is known for increased sensitivity to Vemurafenib in Melanoma, and the gene mutation EGFR T790M for resistance to tyrosine kinase inhibitors. Such associations can be looked up from local/external knowledge bases such as the Catalogue Of Somatic Mutations In Cancer (COSMIC) Database, the Mutations and Drugs Portal (MDP), the Cancer Drug Resistance Database (CancerDR), the Drug Gene Interaction Database (DGIdb) and ClinicalTrials.gov. Additional information on the drugs, such as the side effects, toxicity, mechanism of action, interactions with other drugs and the supporting scientific evidence can be accessed for display. Gathering, summarizing and presenting such information in one single tool can facilitate the design of combinatorial therapy and caution the potential threats of certain drug combinations that should be avoided.

### Example

As a use case example, our Precision Medicine Explorer is used for examining the omic data of an ER+ breast cancer patient. From the top-level view, the oncologist gets a genomic overview of the clinically relevant mutations carried by the patient and the available drug options. As expected, an overexpression of the ESR1 gene was reported with a list of drug options consisting of ER inhibitors. If the oncologist wants to further examine the expression levels of the genes in the ER pathway, she would then add a track for gene expression and filter for a pre-defined panel of ER pathway genes. After inspecting the expression values, she confirmed whether the patient has a hyperactive ER pathway, which could be effectively suppressed by ER inhibitors. She also noticed that the patient carries a known pathogenic mutation in the PIK3CA gene. She clicks on the mutation and checks the allele frequency, function, pathogenicity, call quality, related publications, among other details, and confirmed that the mutation served as a good prognostic biomarker for favorable therapeutic response of PIK3CA inhibitors. After comparing the clinical evidence and possible side effects of the drug options, she decided to administer the two inhibitors with the strongest clinical evidence respectively for suppressing the activities of ER and PIK3CA in combination for treating the patient. Our Precision Medicine Explorer significantly improved the workflow of an oncologist in performing integrative analysis on a patient's omic data for treatment planning.

What is claimed:

1. A computer-implemented method for summarizing and presenting patient-specific multi-omic data in a multilayered format, the method comprising:
a computing device with a graphical user interface,
determining a dataset of files containing patient information by obtaining genomic aberration and other omics data from a patient and storing said data on a non-transitory computer readable storage medium;
determining selection criteria based on the patient dataset;
inputting patient-specific data, by a user interface, onto a processor configured to receive said patient-specific data,
selecting a cohort of samples based on user-defined demographic and phenotypic criteria from a repository of patient or healthy samples, and inputting said demographic and phenotype criteria into said computing device through said graphical user interface;
extracting said cohort genomic aberration and omics data for comparison with the patient of interest based on said demographic and phenotype criteria and inputting said cohort genomic aberration and omics data, by a user interface, onto a processor configured to receive said cohort genomic aberration and omics data;
annotating said patient-specific genomic aberration and omics data in a first layer of said multilayered format, using internal/external knowledge bases, which include information such as mutation impact, population allele frequency, disease association with model of inheritance and drug response;
filtering said patient-specific genomic aberrations and omics data based on user-defined criteria, such as chromosome regions, genes and variant type/function/impact/population allele frequency; and
displaying said patient-specific genomic aberration and omics data in said interactive multi-level format, wherein said multilayer format comprises;
said first layer, said first layer comprising an interactive chromosomal view that summarizes all the clinically relevant or actionable genomic aberrations of said patient by marking them on the genome coordinates, including known drug responses associated with a particular mutation/gene marked next to the mutation/gene accordingly, said first layer further comprising;
a first sub-layer comprising an ideogram view where chromosomes are arranged in a circular format;
a second sub-layer comprising an ideogram view where each chromosome in said first sub-layer is separately displayed in a schematic;
a second layer comprising an interactive intergenic genomic scale where multiple genes are displayed with their expression levels indicated by color;
a third level comprising an interactive genic scale, depicting the structure and functional blocks within a gene, omics data such as methylation levels and gene/exon expression, the 3D protein structure (ribbon plot), with mutations marked and including general information about said gene; and
a fourth level, comprising a molecular scale displaying the molecular sequence and its detailed annotations, such as the nucleotide sequence of the reference genome, the corresponding amino acid sequence in the protein-coding regions, nucleotide/amino acid changes caused by the mutations, exon/gene expression and methylation levels of CpG sites, ChIP-Seq data for histone modification.

2. The method of claim 1, wherein the mulilayered format is a circular or linear multilayered format.

3. The method of claim 1, wherein said obtaining genomic aberration and other omics data from a patient comprises the collection of tissue and blood samples from said patient, performing next-generation sample preparation and DNA/RNA seqeuncing, read alignment and culling of variants and gene expressions.

4. The method of claim 1, wherein said second layer further comprises additional data tracks to add more details, such as methylation, chromatin immunoprecipitation sequencing and assay data which may improve the functional view of genomic aberrations.

5. A non-transitory computer readable storage medium tangibly encoded with computer-executable instructions, that when executed by a processor associated with computing device having a graphical user interface, cause the device to carry out the steps of the method as defined in claim 1.

6. A computer program product, comprising a computer-readable code to be executed by one or more processors when retrieved from a non-transitory computer-readable medium, the computer-readable program code including instructions to:
determine a dataset of files containing patient information by obtaining genomic aberration and other omics data from a patient and storing said data on a non-transitory computer readable storage medium;
receive selection criteria by a user through a graphical user interface, said selection criteria determined by said user based on said patient dataset, and input said patient-specific data, onto a processor configured to receive said patient-specific data,
select a cohort of samples based on user-defined demographic and phenotypic criteria from a repository of patient or healthy samples, and input said demographic and phenotype criteria into said computing device through said graphical user interface;
extract said cohort genomic aberration and omics data for comparison with the patient of interest based on said demographic and phenotype criteria and inputting said cohort genomic aberration and omics data, by a user interface, onto a processor configured to receive said cohort genomic aberration and omics data;
annotate said patient-specific genomic aberration and omics data, using internal/external knowledge bases, which include information such as mutation impact, population allele frequency, disease association with model of inheritance and drug response;
filter said patient-specific genomic aberrations and omics data based on user-defined criteria, such as chromosome regions, genes and variant type/function/impact/population allele frequency; and
display said patient-specific genomic aberration and omics data in said interactive multi-level format, wherein said multilayer format comprises;
a first layer comprising an interactive chromosomal view that summarizes all the clinically relevant or actionable genomic aberrations of said patient by marking them on the genome coordinates, including known drug responses associated with a particular mutation/gene marked next to the mutation/gene accordingly, said first layer further comprising;
a first sub-layer comprising an ideogram view where chromosomes are arranged in a circular format;
a second sub-layer comprising an ideogram view where each chromosome in said first sub-layer is separately displayed in a schematic;
a second layer comprising an interactive intergenic genomic scale where multiple genes are displayed with their expression levels indicated by color;
a third level comprising an interactive genic scale, depicting the structure and functional blocks within a gene, omics data such as methylation levels and gene/exon expression, the 3D protein structure (ribbon plot), with mutations marked and including general information about said gene; and
a fourth level, comprising a molecular scale displaying the molecular sequence and its detailed annotations, such as the nucleotide sequence of the reference genome, the corresponding amino acid sequence in the protein-coding regions, nucleotide/amino acid changes caused by the mutations, exon/gene expression and methylation levels of CpG sites, ChIP-Seq data for histone modification.
